(19) United States
(12) Patent Application Publication
(10) Pub. No.: US 2013/0157270 A1
(43) Pub. Date: Jun. 20, 2013

(54) Marker of Diagnosis and Prognosis in Multiple Sclerosis

(71) Applicants: Horea Rus, Catonsville, MD (US); Cornelia Cudrici, Rockville, MD (US); Cosmin Tegla, Baltimore, MD (US)

(72) Inventors: Horea Rus, Catonsville, MD (US); Cornelia Cudrici, Rockville, MD (US); Cosmin Tegla, Baltimore, MD (US)

(21) Appl. No.: 13/774,854

(22) Filed: Feb. 22, 2013

Related U.S. Application Data
(63) Continuation-in-part of application No. PCT/US2011/001487, filed on Aug. 24, 2011.
(60) Provisional application No. 61/402,121, filed on Aug. 24, 2010.

(51) Int. Cl. C12Q 1/68; G01N 33/567

(52) U.S. Cl. 435/6.11; 435/7.21

(57) ABSTRACT

The present invention provides a method for determining whether an individual with relapsing-remitting multiple sclerosis will suffer a relapse. In the method, measuring the level of Response Gene to Complement (RGC)-32 is measured in the individual, where a significantly lower level of RGC-32 therein indicates that the individual will have or is having a relapse of multiple sclerosis.

### CROSS-REFERENCE TO RELATED APPLICATIONS

This continuation-in-part application claims benefit of priority under 35 U.S.C. §120 of pending international application PCT/US2011/001487, filed Aug. 24, 2011, which claims benefit of priority under 35 U.S.C. §119(e) of provisional application U.S. Ser. No. 61/402,121, filed Aug. 24, 2010, now abandoned, the entirety of both of which are hereby incorporated by reference.

### BACKGROUND OF THE INVENTION

1. Field of the Invention

The present invention relates to the fields of diagnostic and therapeutic neurology. More specifically, the present invention relates to, inter alia, a marker of diagnosis and prognosis in multiple sclerosis.

2. Description of the Related Art

Progression through each phase of the cell cycle is controlled by specific cyclin dependent kinases (CDK) and their interactions with cyclins and CDK inhibitors (CKI). The expression of each cyclin fluctuates throughout the cell cycle, and CKI are down-regulated in response to mitogenic stimulation. Cyclin dependent kinases are a family of serine/threonine protein kinases that are regulated by multiple mechanisms leading to their activation at specific points of the cell cycle. Mitosis is regulated by CDC2 when in complex with cyclin B. Deregulation of the cell cycle is well documented in cancer, and compounds with CDK inhibitory activity have recently entered clinical trials. The expression of CDC2, CDK2 and CDK4 proteins is higher in colon cancer cells than in normal mucosa. Higher levels of cyclins DI, D3, A and E were also found in primary colorectal carcinomas than in the adjacent normal areas. It is also documented that CDC2 kinase activity is increased in colon cancer tissue, but not in normal tissue. CDC2 is mostly present in colon cancer cells positive for phosphorylated Rb protein, and its overexpression is higher in focal carcinomas. The cyclin dependent pathway is, however, complicated and other factors are necessary for proper function and progression through the cell cycle.

One of these other factors, the Response Gene to Complement (RGC)-32 was first cloned in the rat by differential display (1), and subsequently from human brain library (2). Overexpression of RGC-32 is associated with an increase in DNA synthesis, thus leading to the hypothesis that RGC-32 is involved in activation of the cell cycle (1). Experimental evidence indicates that RGC-32 has an important role in cell cycle activation through regulation of CDC2 kinase (2). Overexpression of RGC-32 in human aortic smooth muscle cells (SMC) increased BrdU incorporation and the number of cells progressing into G2/M phase. RGC-32 appears to complex with CDC2/cyclin BI and increase the kinase activity of CDC2. This kinase-enhancing activity requires CDC2 phosphorylation of RGC-32 at Threonine 91. These findings identify RGC-32 as a substrate and regulator of CDC2.

Thus, the Response Gene to Complement (RGC)-32, acts primarily as a cell cycle regulator (1-2). RGC-32 overexpression leads to an increase in DNA synthesis and cell cycle progression from the G1/G0 to G2/M phase (2). Both of these responses can be abolished by transfecting the cells with RGC-32-specific siRNA (3). RGC-32 forms complexes with CDC2 and enhances CDC2 kinase activity (2). Thus, RGC-32 appears to be a previously unrecognized regulator of CDC2, a critical kinase involved in cell cycle activation.

Multiple sclerosis (MS) is a chronic autoimmune inflammatory disease of the central nervous system and is a common cause of persistent disability in young adults. In patients suffering from MS, the immune system destroys the myelin sheet of axons in the brain and the spinal chord, causing a variety of neurological pathologies. In the most common form of MS, Relapsing-Remitting, episodes of acute worsening of neurological function (exacerbations, attacks) are followed by partial or complete recovery periods (remissions) that are free of disease progression (stable). It has been reported that ninety percent of patients with MS initially present with a clinically isolated syndrome because of an inflammatory demyelinating lesion in the optic nerve, brain stem, or spinal cord. About thirty percent of those patients with a clinically isolated syndrome progress to clinically definite MS within 12 months of presentation. The subsequent progression of the disease can vary significantly from patient to patient. The progression can range from a benign course to a classic relapsing—remitting, chronic progressive, or rare fulminant course.

A method for diagnosing MS that facilitates early MS diagnosis and prediction of disease activity (Benign, Moderate and Malignant) would be valuable for both managing the disease and providing counsel for the patient. For example, patients diagnosed early with active course of MS could be offered disease modifying treatments that have recently been shown to be beneficial in early MS.

Current methods for assessment and tracking progress of MS are based on assessment and scoring of patients' function in attacks and accumulated disabilities during the attacks. One assessment used to assess MS is the Expanded Disability Status Scale (EDSS). However, EDSS score system measures the outcome and does not have predict for the progression of the disease. In addition, EDSS scoring can be variable because it is based on a subjective assessment of patient function. Methods for diagnosis can also include tracking brain lesions by Magnetic Resonance Imaging (MRI) or testing Cerebrospinal Fluid (CSF) for Oligo-Clonal Banding (OCB). MRI is a physical method for assessment of brain lesions and is used widely for MS diagnosis. However, it has only very long term predictive value. In addition, the correlation between MRI results and disease activity is poor. Thus, MRI can not be used for short term projections of disease activity or disease management.

Cerebrospinal puncture is an unpleasant invasive procedure that is not suitable for routine use or prognosis. In addition, both methods assess damage only after it has occurred; neither method can predict the onset of attacks or silent, sub-clinical lesions. A further disadvantage in testing for Oligo-Clonal Banding, e.g., in CSF and MRI as a way to diagnose MS is that a negative Oligo-Clonal Banding or MRI will not preclude the existence of MS.

Most patients with MS initially present with a clinically isolated syndrome (CIS). Despite the fact that MS will develop in up to 80% of these patients, the course of the disease is unpredictable at its onset. The disease may remain inactive for many years before the appearance of a second clinical relapse or new lesions on MRI confirm the diagnosis. Because currently available therapy is only partially effective and side effects are common, many neurologists are uncertain whether to treat all such patients with immunomodulators, or to wait until the diagnosis is confirmed by a second clinical event or the appearance of new MRI lesions.

There is a need for a simple serological assay that predicts whether patients with a CIS suggestive of MS or newly diagnosed relapsing remitting MS will have a highly active disease course and therefore require aggressive treatment, or whether they will follow a more benign course that enables such patients to postpone immunomodulatory therapy until necessary. This assay would be also useful in helping the diagnosis of MS. There is also a need for a method that uses objectively assessed markers for diagnosing MS and for predicting disease activity, the onset of attacks or silent lesions in patients suffering from MS.

Little is currently known about the potential role of RGC-32 in autoimmune disorders, including multiple sclerosis (MS). Several studies have demonstrated impaired apoptosis of T cells in multiple sclerosis patients (4-6). Furthermore, relapses may be associated with the persistent presence of myelin-activated T cells resulting from impaired T-cell apoptosis (4-6). T-cell apoptosis in both experimental allergic encephalomyelitis (EAE) and multiple sclerosis is regulated in part by the Fas-FasL system (6), and ex vivo studies have demonstrated an increased resistance of T cells to Fas-mediated apoptosis during multiple sclerosis relapses (7). In addition, FasL expression has been found to be low during relapses, consistent with the increased resistance of the T cells to apoptosis (8). FasL expression on T cells is regulated by multiple factors, including the CDC2/cyclin B1 complex (9). Since RGC-32 binds to CDC2/cyclin B1complex and up-regulates its activity, it is possible that RGC-32 is involved in regulating T-cell survival by modulating the expression of FasL. Preliminary studies have shown that RGC-32 is expressed by CD3⁺ as well as CD4⁺ T cells from peripheral blood (PB) and in brain tissue from MS patients (10-11).

Thus, there is a continued need in the art for improved methods and therapies to diagnose and treat multiple sclerosis and autoimmune diseases. The present invention fulfills this long standing need and desire in the art.

### SUMMARY OF THE INVENTION

The present invention is directed to a method of determining whether an individual with relapsing-remitting multiple sclerosis will suffer a relapse, comprising the step of measuring the level of Response Gene to Complement-32 in said individual, wherein a significantly lower level of Response Gene to Complement-32 in said individual indicates that said individual will have or is having a relapse of multiple sclerosis.

In another embodiment, the present invention provides a method of determining whether an individual with relapsing-remitting multiple sclerosis will respond positively to a pharmacologic treatment for multiple sclerosis, comprising the step of measuring the level of Response Gene to Complement (RGC)-32 in said individual, wherein the absence of a significant decrease in the level of Response Gene to Complement-32 in said individual indicates that said individual will respond positively or is responding positively to a pharmacologic treatment for multiple sclerosis.

In yet another embodiment, the present invention provides a method of determining whether an individual with relapsing-remitting multiple sclerosis will suffer a relapse, comprising the steps of measuring the level of Response Gene to Complement-32 in said individual, and measuring FasL levels in said individual, wherein significantly lower levels of Response Gene to Complement-32 and FasL levels in said individual indicates that said individual will have or is having a relapse of multiple sclerosis.

In yet another embodiment, the present invention provides a method of determining whether an individual with relapsing-remitting multiple sclerosis is in a period of stable disease or is not at risk for relapse of multiple sclerosis, comprising the step of measuring the level of Response Gene to Complement-32 in said individual, wherein a significantly higher level of Response Gene to Complement-32 in said individual indicates that said individual is in a period of stable disease or is not at risk for relapse of multiple sclerosis.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 depicts the expression of RGC-32 mRNA in MS patients and controls. The expression of mRNA was measured by real time PCR and expressed as ratio L13. A statistically significant decrease of RGC-32 was found in patients with relapses when compared with stable MS patients and controls. RGC-32 levels were found to be significantly higher in stable MS patients.

FIGS. 2A-2D show the expression of RGC-32 on inflammatory cells in MS brain. MS plaques cryostat sections were immunostained by the ABC method using an rabbit IgG anti-RGC-32. FIGS. 2A-2B show that perivascular inflammatory cells were positive for RGC-32 at the arrows. FIG. 2C shows that RGC-32 (light gray deposits) was co-localized with CD3 (dark gray deposits) by double staining at the arrowheads in the parenchyma of an MS plaque; FIG. 2D is the control for the immunoperoxidase reaction.

FIGS. 3A-3C demonstrate expression of RGC-32 in CD4⁺ T-cells form MS patients and controls. FIG. 3A shows that RGC-32 was present in CD4⁺ cells mostly in the cytoplasm and less in the nucleus (×400, inset: ×1000). FIG. 3B shows that Control of the immunoperoxidase reaction using isotype control instead of the primary antibody was negative (×400). FIG. 3C shows that significantly higher number of CD4⁺ cells expressing RGC-32 was found in stable MS patients when compared to controls (p<0.01) and to MS patients with relapses (p<0.003).

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device or method described herein can be implemented with respect to any other device or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “contacting” refers to any suitable method of bringing a compound or a composition into contact with a cell. For in vivo applications, any known method of administration is suitable as described herein.

As used herein, the term “subject” refers to any human or non-human recipient of the composition described herein.

The present invention is directed to a method of determining whether an individual with relapsing-remitting multiple sclerosis will suffer a relapse, comprising the step of measuring the level of Response Gene to Complement-32 in said individual, wherein a significantly lower level of Response Gene to Complement-32 in said individual indicates that said individual will have or is having a relapse of multiple sclerosis. A person having ordinary skill in this art would readily recognize that testing of level of Response Gene to Complement-32 in an individual with relapsing-remitting multiple sclerosis can be undertaken in various time periods. For example, the levels of Response Gene to Complement-32 may be measured daily, weekly or monthly.

In one preferred aspect of this method of the present invention, levels of said Response Gene to Complement-32 are measured on a daily basis. In this embodiment, a significantly lower level in said Response Gene to Complement-32 from one day to the next indicates that said individual will have or is having a relapse of multiple sclerosis.

In another preferred aspect of this method of the present invention, levels of said Response Gene to Complement-32 are measured on a weekly basis. In this embodiment, a significantly lower level in said Response Gene to Complement-32 from one week to the next indicates that said individual will have or is having a relapse of multiple sclerosis. In another preferred aspect of this method of the present invention, levels of said Response Gene to Complement-32 are measured on a monthly basis. In this embodiment, a significantly lower level in said Response Gene to Complement-32 from one month to the next indicates that said individual will have or is having a relapse of multiple sclerosis. As is well known, the levels of Response Gene to Complement-32 may be measured at the protein or mRNA level. For example, Response Gene to Complement-32 mRNA may measured by real time PCR or an oligonucleotide array. In one form, the Response Gene to Complement-32 mRNA is measured in peripheral blood mononuclear cells. In a non-limiting example, the peripheral blood mononuclear cells may be CD4⁺ T-cells.

In a further aspect of this embodiment of the present invention, the method further comprises the step of measuring FasL levels, wherein significantly lower FasL levels indicates that said individual is suffering a relapse of multiple sclerosis. Within the context of the methods of the present invention, the term “significantly” refers to a statistically significant difference from one measurement to the next. Thus, a “significant” reduction or increase is one that reaches statistical relevance or significance.

The present invention is directed to a method of determining whether an individual with relapsing-remitting multiple sclerosis will respond positively to a pharmacologic treatment for multiple sclerosis, comprising the step of measuring the level of Response Gene to Complement (RGC)-32 in said individual, wherein the absence of a significant decrease in the level of RGC-32 in said individual indicates that said individual will respond positively or is responding positively to a pharmacologic treatment for multiple sclerosis. A person having ordinary skill in this art would readily recognize that testing of level of Response Gene to Complement-32 in an individual with relapsing-remitting multiple sclerosis can be undertaken in various time periods. For example, the levels of Response Gene to Complement-32 may be measured daily, weekly or monthly.

In one preferred aspect of this method of the present invention, levels of said Response Gene to Complement-32 are measured on a daily basis. In this embodiment, a significantly lower level in said Response Gene to Complement-32 from one day to the next indicates that said individual will respond positively or is responding positively to a pharmacologic treatment for multiple sclerosis.

In another preferred aspect of this method of the present invention, levels of said Response Gene to Complement-32 are measured on a weekly basis. In this embodiment, a significantly lower level in said Response Gene to Complement-32 from one week to the next indicates that said individual will respond positively or is responding positively to a pharmacologic treatment for multiple sclerosis.

In yet another preferred aspect of this method of the present invention, levels of said Response Gene to Complement-32 are measured on a monthly basis. In this embodiment, a significantly lower level in said Response Gene to Complement-32 from one month to the next indicates that said individual will respond positively or is responding positively to a pharmacologic treatment for multiple sclerosis. As is well known, the levels of Response Gene to Complement-32 may be measured at the protein or mRNA level. For example, Response Gene to Complement-32 mRNA may measured by real time PCR or an oligonucleotide array. In one form, the Response Gene to Complement-32 mRNA is measured in peripheral blood mononuclear cells. In a non-limiting example, the peripheral blood mononuclear cells may be CD4⁺ T-cells.

In a further aspect of this embodiment of the present invention, the method further comprises the step of measuring FasL levels, wherein significantly lower FasL levels indicates that said individual will respond positively or is responding positively to a pharmacologic treatment for multiple sclerosis. A person having ordinary skill in this art would readily recognize that the methods of the present invention would be useful for any pharmacologic treatment. In a preferred aspect of this embodiment of the present invention, the pharmacologic treatment is glatiramer acetate.

The present invention is further directed to a method of determining whether an individual with relapsing-remitting multiple sclerosis is in a period of stable disease or is not at risk for relapse of multiple sclerosis, comprising the step of measuring the level of Response Gene to Complement-32 in said individual, wherein a significantly higher level of Response Gene to Complement-32 in said individual indicates that said individual is in a period of stable disease or is not at risk for relapse of multiple sclerosis. This method would be useful to clinicians in a variety of ways. For example, following a relapse of multiple sclerosis in an individual, the levels of Response Gene to Complement-32 may be measured daily, weekly or monthly to monitor the status of the individual and to determine when the individual is beginning to relapse. Methods of measuring the level of said Response Gene to Complement-32 at the protein or mRNA level is described above. This method may further comprising the step of measuring FasL levels, wherein significantly higher FasL levels indicates that said individual is in a period of stable disease or is not at risk for relapse of multiple sclerosis.

The Response Gene to Complement 32 (RGC-32) is a molecule that plays an important role in cell proliferation and death. The present invention found significantly lower levels of RGC-32 mRNA in multiple sclerosis (MS) patients with relapses than in patients who were clinically stable or in control subjects. Furthermore, the present invention shows that the decrease in RGC-32 levels signals the onset of a relapse, and hence is a biomarker that could be used to predict disease activity and predict the response to MS therapy, such as with glatiramer acetate. Patients with no further relapse MS therapy will maintain high levels of RGC-32 mRNA. On the other hand, those with relapses experience a drop in RGC-32 mRNA expression preceding the clinical relapse. Those with persistently low levels of RGC-32 are expected to respond less well to therapy with glatiramer acetate.

The following example(s) are given for the purpose of illustrating various embodiments of the invention and are not meant to limit the present invention in any fashion.

### EXAMPLE 1

### Methods

### Collection of PBMC and Isolation of RNA and CD4+ Cells

Peripheral blood mononuclear cells (PBMC) are collected using BD Vacutainer CPT tubes (Becton Dickinson, Franklin Lakes, N.J.). Mononuclear cells are isolated from fresh blood according to the manufacturer's protocol. In the case of PBMC, RNA isolation and cell lysate preparation for protein analysis are performed the same day (12). For purification of total RNA from PBMC, RNeasy Kits (Qiagen, Santa Clarita, Calif.) are used. Generally,

### Isolation of CD4+ Cells

PBMCs were collected using BD Vacutainer CPT tubes (Becton Dickinson, Franklin Lakes, N.J.). The PB CD4+ cells were isolated by negative selection using magnetic microbeads (Miltenyi Biotec, Auburn, Calif.). The purity of human T-cell subsets obtained using this technique has been consistently >95%, as assessed by FACS analysis (16).

### Real-Time Quantitative PCR

RNA is reverse-transcribed to cDNA using reverse transcriptase reagents according to the manufacturer's protocol (Perkin Elmer, Foster City, Calif.). Real-time PCR is performed using a One step PCR system (Perkin Elmer). Real-time PCR is performed according to the manufacturer's protocol using the FastStart SYBR Green master mix (Roche, Indianapolis, Ind.). Specific primers (forward and reverse) were designed (TIB BioMol, Adelphia, N.J.) to determine mRNA expression of RCG-32 (Forward: 5′-AGGAACAGCTTCAGCTTCAG-3′, SEQ ID NO: 1; Reverse: 5′-GCTAAAGTTTTGTCAAGATCAGCA-3′, SEQ ID NO: 2), FasL (Forward: 5′-GCCCATTTAACAGGCAAGTC-3′, SEQ ID NO: 3; Reverse: 5′-ATCACAAGGCCACCCTTCTT-3′, SEQ ID NO: 4), CDC2 (Forward: 5′-TTTTCAGAGCTTTGGGCACT-3′, SEQ ID NO: 5; Reverse: 5′-AGGCTTCCTGGTTTCCATTT-3′, SEQ ID NO: 6), and L13 (Forward: 5′-CGTGCGTCTGAAGCCTACA-3′, SEQ ID NO: 7; Reverse: 5′-GGAGTCCGTGGGTCTTGAG-3′, SEQ ID NO: 8). These primers are used at a concentration of 200 nM/l. L13 mRNA expression is used for sample normalization.

### Western Blot Analysis

PBMC are lysed in RIPA buffer and processed as previously described (11). Whole-cell lysates (total protein=10-30 μg) are analyzed by 12% SDS-PAGE, followed by western blotting. Each membrane is used for RGC-32 expression. In addition the levels of protein expression for FasL and CDC2 are tested by western blotting using specific antibodies (12). Anti-rabbit HRP-conjugated antibody are used as the secondary antibody, and the signals are visualized by enhanced chemiluminescence (Pierce, Rockford, Ill.) and autoradiography Blots are stripped and probed again for beta-actin expression, which serves as a control for normalization.

### EXAMPLE 2

### Expression of RGC-32

Peripheral Mononuclear Cells (PBMC) from Relapsing Remitting (RR) MS Patients and Controls

Initially, the expression of RGC-32 mRNA was examined using cDNA profiling arrays (Clontech, Mountain View, Calif.). RGC-32 expression was significantly lower in CD3+ T cells from patients with MS relapses than in healthy controls (10-11). In the next step, a total of 20 patients (10 patients with inactive MS and 10 with relapses) and 20 healthy, age-, gender- and race-matched controls were examined. In these patients, RGC-32 expression was analyzed in unstimulated PBMCs under conditions as close as possible to the in vivo situation because alterations in these cells are likely to have predictive value for clinical exacerbations.

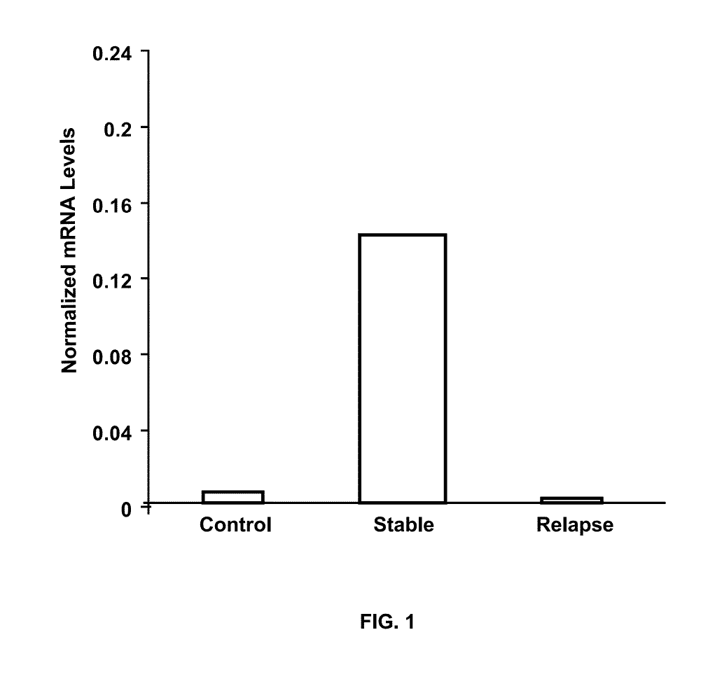

Expression of RGC-32 and L13 mRNA, a housekeeping gene, was measured by real-time PCR. In addition, the expression of FasL and CDC2 mRNA was assessed, since the expression of their genes is regulated by RGC-32. Significantly lower levels of RGC-32 mRNA were found in MS patients with relapses than in patients who were clinically stable or in control subjects (FIG. 1). In addition, MS patients in remission had higher levels of RGC-32 than did either the controls (p<0.01) or patients with relapses (p<0.01). Similar results were obtained for FasL mRNA expression. Patients with relapses had significant lower FasL levels than did control subjects (p<0.01) or stable MS patients (p<0.01). On the other hand, CDC2 mRNA levels were lower in MS patients with relapses than in stable MS patients, but this difference did not reach statistical significance (10-11).

### Multiple Sclerotic Brain

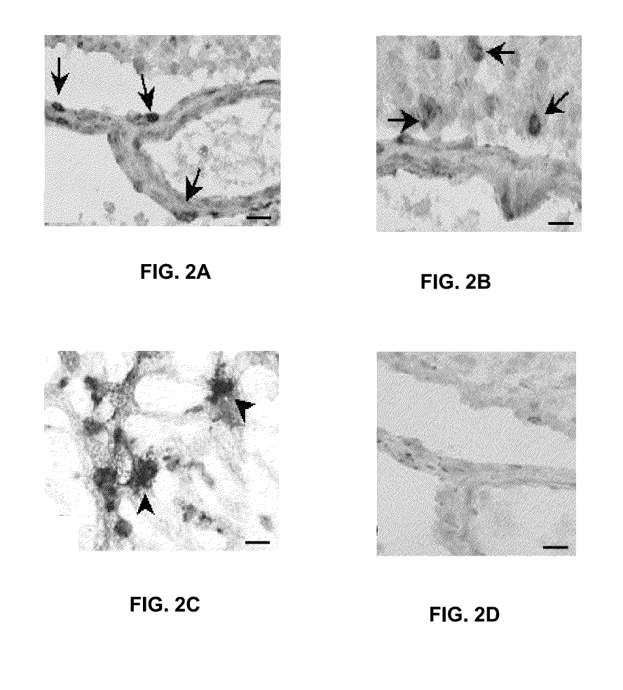

Using indirect immunoperoxidase staining, the present invention shows that RGC-32 is expressed in both acute and chronic active lesions. The RGC-32 deposition extended from the MS plaques to normal adjacent white and gray matter lesions (FIGS. 2A-2D). By using double-staining immunohistochemistry, it was demonstrated that some of CD3⁺ cells in MS plaques co-localized with RGC-32. The RGC-32 also co-localized with CD68⁺ macrophages (10-11).

FIG. 1 shows the expression of RGC-32 mRNA in MS patients and controls. The expression of mRNA was measured by real time PCR and expressed as ratio L13. A statistically significant decrease of RGC-32 was found in patients with relapses when compared with stable MS patients and controls. RGC-32 levels were found to be significantly higher in stable MS patients.

FIGS. 2A-2B shows the expression of RGC-32 on inflammatory cells in MS brain. MS plaques cryostat sections were immunostained by the ABC method using an rabbit IgG anti-RGC-32. FIGS. 2A and 2B show that perivascular inflammatory cells were positive for RGC-32 compared to control FIG. 2C shows that RGC-32 (light gray deposits) were co-localized with CD3 (dark grey deposits) by double staining at the arrowheads in the parenchyma of an MS plaque compared to control for the immunoperoxidase reaction.

TABLE 1

| | | | | | | | Parenchymal |
|---|---|---|---|---|---|---|---|
| | | | | No. | Lesional | Perivascular | infiltrate/ |
| Case | Sex | Age | Lesion types | of lesions | activity | infiltrate | microglia |
| 1 | F | 47 | Frontal | 3 | Chronic | +++ | + |
| | | | plaque | | active | | |
| | | | | | NLWM | ++ | ++ |
| | | | | | NLGM | + | ++ |
| 2 | M | 50 | Frontal | 1 | NLGM | +++ | − |
| | | | plaque | | | | |
| 3 | M | 50 | Temporal | 3 | Acute | ++ | +++ |
| | | | Plaque | | | | |
| | | | | | NLWM | ++ | ++ |
| | | | | | NLGM | + | + |
| 4 | M | 50 | Occipital | 1 | NLWM | +/++ | +++ |
| | | | Plague | | | | |
| 5 | F | 51 | Frontal | 3 | Acute | ++ | ++ |
| | | | plaque | | | | |
| | | | | | NLWM | ++ | +++ |
| | | | | | NLGM | ++ | ++ |
| 6 | F | 51 | Occipital | 3 | Chronic | +++ | +++ |
| | | | Plaque | | active | | |
| | | | | | NLWM | +++ | +++ |
| | | | | | NLGM | ++ | + |
| 7 | F | 38 | Parietal | 3 | Chronic | ++ | ++ |
| | | | plaque | | active | | |
| | | | | | NL MW | + | ++ |
| | | | | | NL GW | + | + |
| 8 | F | 38 | Occipital | 3 | Chronic | +++ | +++ |
| | | | Plaque | | active | | |
| | | | | | NLWM | +++ | +++ |
| | | | | | NLGM | ++ | ++ |

NLAM: non-lesional white matter,
NLGM: non-lesional gray matter,

As shown in the Table 1 above, the present invention demonstrates that inflammatory cells expressing RGC-32 cross the brain blood barrier during acute events and infiltrate multiple sclerosis lesions in patients with acute and chronic active multiple sclerosis. This data also supports the hypothesis that the changes in RGC-32 mRNA that are related to clinical activity are a result of the migration of inflammatory cells to the central nervous system.

The present invention shows that RGC-32 can serve as a biomarker for relapse in multiple sclerosis (MS) patients and that administration of glatiramer acetate, is associated with alterations in RGC-32 expression. Specifically, there are correlations between the level of RGC-32 and the response of MS patients to treatment. The present invention establishes a role for RGC-32 in multiple sclerosis as a possible biomarker of relapses and as a predictor of response to treatment. By measuring the level of RGC-32 expression in PBMCs, a person having ordinary skill in this art could use such analyses as a test for predicting relapses and response to multiple sclerosis drugs, such as glatiramer acetate (Copaxone) therapy in clinical practice.

RGC-32 involvement in multiple sclerosis can be further addressed in a prospective clinical study and in studies designed to look for correlations between the levels of the RGC-32 and disease activity.

### Expression of RGC-32 in CD4+ T-Cells of RR MS Patients and Controls

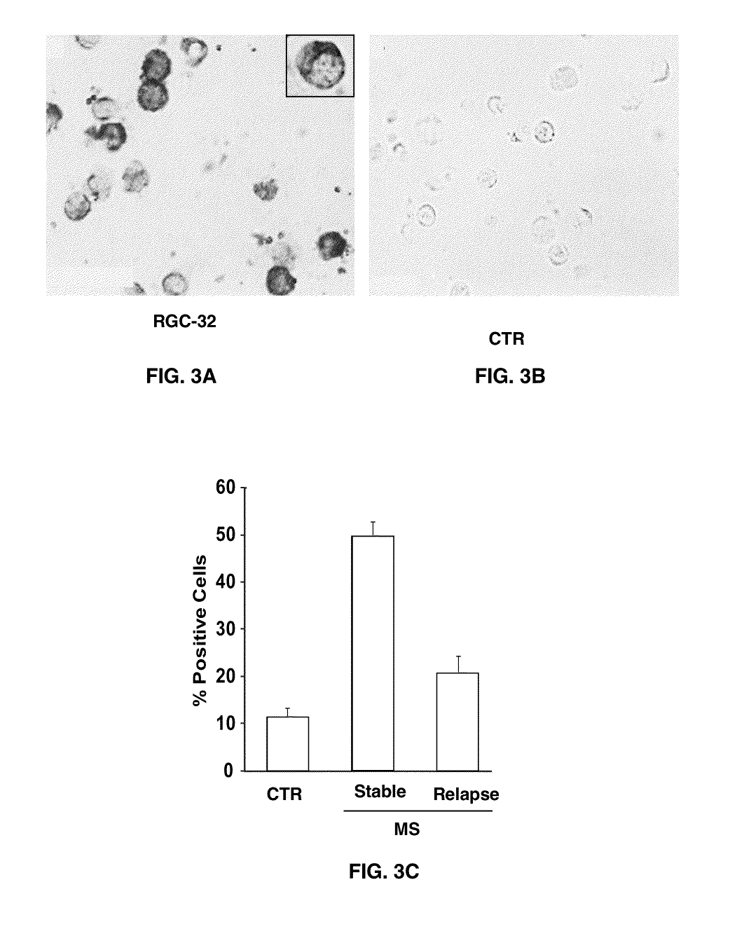

CD4⁺ T-cells were isolated from PB cells by negative selection, deposited on glass slides in a cytospin centrifuge and then fixed in acetone:methanol (2:1). RGC-32 expression was determined by immunocytochemistry as described in Example 1. RGC-32 was present in CD4+ cells mostly in the cytoplasm and sometimes in the nucleus (FIG. 3A). Controls of immunoperoxidase reaction were negative (FIG. 3B). Significantly higher number of CD4+ cells expressing RGC-32 was found in stable MS patients when compared to controls (p<0.001) and to patients with relapses (p<0.003) (FIG. 3C). The expression pattern of RGC-32 protein in CD4+ is similar to that of mRNA in PBMCs with low levels of expression during relapses when compared to stable MS patients.

### EXAMPLE 3

### Glatiramer Acetate Treatment of Multiple Sclerosis

### In Vivo Study

In a study, multiple sclerosis patients are treated with glatiramer acetate (20 mg) by subcutaneous injection every day and followed for up to 2 years. To increase the likelihood of including patients who will have relapses during this study, patients presenting with MS relapses are preferentially admitted into the study.

### Samples

PBMC are obtained from patients at 0, 2, 4, 26, 52, 78, and 104 weeks, at the time of their outpatient visits. PBMC are also obtained from controls at 0 weeks. Protein cell lysates and RNA samples are first tested for RGC-32 expression in patients with stable disease to allow assessment of the consistency of the pattern of these markers in stable MS. These data will provide baseline data for comparison when patients' measurements are obtained at later time points.

A total of 30 patients with relapsing remitting MS is enrolled in the study. The patients are primarily recruited from the University of Maryland Multiple Sclerosis Center, which currently follows more than 2000 patients with MS. Patients are followed to allow adequate statistical analysis for the study. Criteria for inclusion in the study is: (i) age 18 to 55 years; (ii) fulfillment of McDonald criteria for definite MS (13-14); (iii) relapsing-remitting course; (iv) having newly diagnosed MS, or MS not treated with currently used immunomodulatory drugs (IFNb or glatiramer acetate) for 3 months prior to study entry; (v) no exacerbations in the 4 weeks before the study; (vi) no iv or po steroids for 4 weeks prior the study enrollment; (vii) no treatment with Tysabri, mitoxantrone, cyclophosphamide, or investigational drugs; and (viii) a disability score of 0-5.5 as defined by the expanded disability status scale (EDSS) (15). Exclusion criteria will be: (i) a history of autoimmune disorders, vascular disease, or active acute or chronic infections; (ii) use of antibiotics in the last 30 days; (iii) a history of intracranial or intraspinal tumor or metabolic myelopathy; (iv) a history of alcohol or drug abuse.

Twenty healthy, age-, gender-, and race-matched controls are enrolled in the study from the outpatient services and staff at the University of Maryland. Exclusion criteria for controls are: the presence of (i) overt acute or chronic disease(s) or (ii) other autoimmune disease(s).

### Interventions

All MS patients receive Copaxone (glatiramer acetate, 20 mg) injected subcutaneously every day for 2 years. MRI examinations are not done systematically in this study but are used in selected cases of significant worsening (more than 1 grade on the EDSS scale).

### Clinical Evaluation

Clinical evaluation of patients is performed at 0, 2, 4, 26 and 52, 78 and 104 weeks, at the time of their outpatient visits. Patients with symptoms suggestive of a clinical relapse are asked to call the University of Maryland Multiple Sclerosis Center. Clinical relapse is defined as substantial worsening of pre-existing symptoms or appearance of new neurological deficits in the absence of fever or infections lasting more than 24 hours. A standardized consultation form and the EDSS is completed at each visit. Clinical records, consultation reports, and inpatient records are reviewed to ensure that the data obtained are complete. In the case of some patients with relapse, the administration of 1 g of Solu-Medrol i.v. for 3 days is necessary. A predisone taper may be also used after i.v. Solu-Medrol in certain cases. In such cases, blood samples are obtained prior to Solu-Medrol treatment.

### Laboratory Testing

RGC-32 mRNA expression is assessed by real-time PCR and western blotting and these results with FasL and CDC2 expression are correlated. In addition, RGC-32 expression is correlated with clinical parameters as mentioned above and with the brain MRI activity.necessary. A predisone taper may be also used after i.v. Solu-Medrol in certain cases.

In such cases, blood samples are obtained prior to Solu-Medrol treatment.

Patients with no further relapse on glatiramer acetate maintain high levels of RGC-32 mRNA. On the other hand, those with relapses should experience a drop in RGC-32 mRNA expression preceding the clinical relapse. Those with persistently low levels of RGC-32 are expected to respond less well to therapy.

Statistical analysis is performed by using the SPSS 11.5 package (SPSS Inc, Chicago, Ill.) for MS-Windows. Comparisons between healthy controls and the different clinical forms of MS are performed with the Kruskal-Wallis test. If significant differences (p>0.05) were found, a Mann-Whitney's test was then used to test for significant differences between two groups.

The association between biological markers and EDSS is analysed using categorical regression analysis (CATREG version 2.1). Categorical regression quantifies categorical data by assigning numerical values to the categories, resulting in an optimal linear regression equation for the transformed variables. CATREG extends the standard approach by simultaneously scaling nominal, ordinal, and numerical variables. Using non-linear transformations allows variables to be analysed at a variety of levels to find the best-fitting model. In view of multiple testing, stringent Bonferroni correction is applied.

The following references were cited herein:
1. Badea et al. 1998. J Biol Chem, 273:26977-26981 (1998)
2. Badea et al. J Biol Chem, 277:502-508 (2002).
3. Fosbrink et al. Exp Mol Pathol, 86:87-94 (2009).
4. Zipp et al. J Neuroimmunol, 86:151-154 (1998).
5. Sharief, M. K., J Neuroimmunol, 109:236-243 (2000).
6. Aktas et al. Neuroscientist, 12:305-316 (2006).
7. Okuda et al. J Neuroimmunol, 171:163-170 (2006).
8. Lopatinskaya et al. J Neuroimmunol, 138:123-131 (2003).
9. Torgler et al. J Biol Chem, 279:37334-37342 (2004).
10. Tegla et al. Multiple Sclerosis, 15:S189 (2009).
11. Tegla et al. Exp. Mol. Pathol., 34:17-28 (2013).
12. Niculescu et al. J Immunol, 158:4405-4412 (1997).
13. McDonald, et al. Ann Neurol, 50:121-127 (2001).
14. Polman et al. Ann Neurol, 58:840-846 (2005).
15. Kurtzke, J. F. Neurology, 33:1444-1452 (1983).
16. Rus et al. P NATL ACAD SCI USA, 102:11094-11099 (2005).

One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for determining whether an individual with relapsing-remitting multiple sclerosis will suffer a relapse, comprising the step of:
measuring the level of Response Gene to Complement-32 in said individual, wherein a significantly lower level of Response Gene to Complement-32 in said individual indicates that said individual will have or is having a relapse of multiple sclerosis.

2. The method of claim 1, wherein said Response Gene to Complement-32 is measured daily, weekly or monthly.

3. The method of claim 1, wherein levels of said Response Gene to Complement-32 are measured on a daily basis in said individual.

4. The method of claim 1, wherein a significantly lower level in said Response Gene to Complement-32 from one day to the next indicates that said individual will have or is having a relapse of multiple sclerosis.

5. The method of claim 1, wherein a significantly lower level in said Response Gene to Complement-32 from one week to the next indicates that said individual will have or is having a relapse of multiple sclerosis.

6. The method of claim 1, wherein said level of said Response Gene to Complement-32 is measured at the protein or mRNA level.

7. The method of claim 6, wherein said Response Gene to Complement-32 mRNA is measured in peripheral blood mononuclear cells.

8. The method of claim 7 wherein the peripheral blood mononuclear cells are CD4+ T-cells.

9. The method of claim 1, wherein a significantly lower level in said Response Gene to Complement-32 from one month to the next indicates that said individual will have or is having a relapse of multiple sclerosis.

10. The method of claim 1, further comprising the step of:
measuring FasL levels, wherein significantly lower FasL levels indicates that said individual is suffering a relapse of multiple sclerosis.

11. A method for determining whether an individual with relapsing-remitting multiple sclerosis will respond positively to a pharmacologic treatment for multiple sclerosis, comprising the step of:
measuring the level of Response Gene to Complement (RGC)-32 in said individual, wherein the absence of a significant decrease in the level of RGC-32 in said individual indicates that said individual will respond positively or is responding positively to a pharmacologic treatment for multiple sclerosis.

12. The method of claim 11, wherein said Response Gene to Complement-32 is measured daily, weekly or monthly.

13. The method of claim 12, wherein levels of said Response Gene to Complement-32 are measured on a daily basis in said individual.

14. The method of claim 12, wherein an absence of a significant decrease in the level of RGC-32 in said individual indicates that said individual will respond positively or is responding positively to a pharmacologic treatment for multiple sclerosis.

15. The method of claim 12, wherein the absence of a significant decrease in the level of RGC-32 from one week to the next in said individual indicates that said individual will respond positively or is responding positively to a pharmacologic treatment for multiple sclerosis.

16. The method of claim 12, wherein said level of said Response Gene to Complement-32 is measured at the protein or mRNA level.

17. The method of claim 16, wherein said Response Gene to Complement-32 mRNA is measured in peripheral blood mononuclear cells.

18. The method of claim 17, wherein peripheral blood mononuclear cells are CD4+ T-cells.

19. The method of claim 11, wherein the absence of a significant decrease in the level of said Response Gene to Complement-32 from one month to the next indicates that said individual will respond positively or is responding positively to a pharmacologic treatment for multiple sclerosis.

20. The method of claim 11, further comprising the step of:
measuring FasL levels, wherein the absence of a significant decrease in lower FasL levels indicates that said individual will respond positively or is responding positively to a pharmacologic treatment for multiple sclerosis.

21. The method of claim 11, wherein said pharmacologic treatment is glatiramer acetate.

22. A method for determining whether an individual with relapsing-remitting multiple sclerosis is in a period of stable disease or is not at risk for relapse of multiple sclerosis, comprising the step of:
measuring the level of Response Gene to Complement-32 in said individual, wherein a significantly higher level of Response Gene to Complement-32 in said individual indicates that said individual is in a period of stable disease or is not at risk for relapse of multiple sclerosis.

23. The method of claim 22, wherein said Response Gene to Complement-32 is measured daily, weekly or monthly.

24. The method of claim 22, wherein levels of said Response Gene to Complement-32 are measured on a daily basis in said individual.

25. The method of claim 22, wherein said level of said Response Gene to Complement-32 is measured at the protein or mRNA level.

26. The method of claim 25, wherein said Response Gene to Complement-32 mRNA is measured by real time PCR or an oligonucleotide array.

27. The method of claim 25, wherein said Response Gene to Complement-32 mRNA is measured in peripheral blood mononuclear cells.

28. The method of claim 22, further comprising the step of:
measuring FasL levels, wherein significantly higher FasL levels indicates that said individual is in a period of stable disease or is not at risk for relapse of multiple sclerosis.


SEQUENCE LISTING (8 sequences)

SEQ ID NO: 1 - DNA, 20 bp, Artificial Sequence
  forward primer for RCG-32 for PCR
aggaacagcttcagcttcag

SEQ ID NO: 2 - DNA, 24 bp, Artificial Sequence
  reverse primer for RCG-32 for PCR
gctaaagttttgtcaagatcagca

SEQ ID NO: 3 - DNA, 20 bp, Artificial Sequence
  forward primer for FasL for PCR
gcccatttaacaggcaagtc

SEQ ID NO: 4 - DNA, 20 bp, Artificial Sequence
  reverse primer for FasL for PCR
atcacaaggccacccttctt

SEQ ID NO: 5 - DNA, 20 bp, Artificial Sequence
  forward primer for CDC2 for PCR
ttttcagagctttgggcact

SEQ ID NO: 6 - DNA, 20 bp, Artificial Sequence
  reverse primer for CDC2 for PCR
aggcttcctggtttccattt

SEQ ID NO: 7 - DNA, 19 bp, Artificial Sequence
  forward primer for L13 for PCR
cgtgcgtctgaagcctaca

SEQ ID NO: 8 - DNA, 19 bp, Artificial Sequence
  reverse primer for L13 for PCR
ggagtccgtgggtcttgag